(19) United States
(12) Patent Application Publication
(10) Pub. No.: US 2018/0311276 A1
(43) Pub. Date: Nov. 1, 2018

(54) Method and Apparatus for Treating Oral Disease in Animals

(71) Applicant: James C. Richards, Sudbury, MA (US)

(72) Inventor: James C. Richards, Sudbury, MA (US)

(21) Appl. No.: 15/806,025

(22) Filed: Nov. 7, 2017

Related U.S. Application Data
(63) Continuation-in-part of application No. 15/260,125, filed on Sep. 8, 2016.
(60) Provisional application No. 62/215,350, filed on Sep. 8, 2015.

(51) Int. Cl. A61K 33/18; A01K 7/06; A01K 11/00; A61Q 11/00; A61P 31/04; A61K 8/20

(52) U.S. Cl. CPC A61K 33/18; A01K 7/06; A61K 8/20; A61Q 11/00; A61P 31/04; A01K 11/006

(57) ABSTRACT

Embodiments of the present invention are directed to methods and apparatus for the treatment of plaque, halitosis and other oral diseases in animals such as dogs. The methods and apparatus feature the administration of molecular iodine at a concentration that is effective and accepted by animals. Clinical trials show that the dose should not be below 0.5 micro grams/mL and not above 2.0 micrograms/mL. Concentrations outside this range are not effective on the low side and refused by the animal on the high side.

This is a continuation-in-part of application Ser. No. 15/260,125 filed Sep. 8, 2016 which claimed priority to provisional application No. 62/215,350 filed Sep. 8, 2015. Applications Ser. No. 15/260,125 and 62/215,350 are hereby incorporated by reference in their entireties.

### FIELD OF THE INVENTION

The present invention relates generally to the field of animal care and animal oral health.

### BACKGROUND OF THE INVENTION

Molecular iodine has been used to treat infections in animals and humans. Its use in animal care and animal oral health has been difficult. It would be useful to have effective therapies for the treatment of diseases of the oral cavity in animals.

### SUMMARY OF THE INVENTION

Embodiments of the present invention are directed to methods and apparatus for administering an effective amount of molecular iodine (I₂) to treat oral disease in animals. As used herein, the term “molecular iodine” refers to the diatomic form of iodine (I₂) and does not comprise other forms of iodine, such as ionic iodine or iodine reacted with other molecules. As used herein, the term “oral disease” refers to infectious diseases of the mouth, oral cavity, including teeth, gums and lips. The word “treat” is used to denote reducing or mitigating the effect of the disease and/or prevent the disease from occurring in a prophylactic sense.

One embodiment of the present invention directed to a method of treating an animal for an oral disease comprises the steps of placing molecular iodine in an aqueous solution in a concentration of 0.01 to 1.0 micrograms per milliliter and allowing the animal to drink of the solution.

Embodiments of the present method are suited for the treatment of halitosis and plaque in animals. The concentration of the solution comprising molecular iodine is below the threshold of the animal's ability to smell and below an animal's threshold for adverse taste.

One embodiment of the present invention features a concentration of 0.5 to 1.0 micrograms per milliliter. Such a concentration is useful for treating acute symptomatic animals. One embodiment features solution is made as the animal approaches a solution dispensing station.

One embodiment of the method features the step of providing a supply of water over time, purging the solution and regenerating the solution.

One embodiment of the present invention features an apparatus for dispensing molecular iodine to an animal. The apparatus comprises an animal detector for sensing the presence or approach of an animal and providing a dispensing signal to a dispensing means. The apparatus further comprises a water supply for containing an aqueous solution for consumption by oral means by the animal. The apparatus further comprises a reservoir of molecular iodine in fluid communication with dispensing means. The apparatus further comprises dispensing means in signal communication with the animal detector and in fluid communication with the reservoir and the water supply. The dispensing means measures an amount of molecular iodine from the reservoir to the water supply and in response to the dispensing signal forming an aqueous solution having a concentration of molecular iodine not exceeding 1.0 micrograms per milliliter.

A further embodiment of the present invention is directed to a formulation for treating the oral cavity of an animal. The formulation comprises molecular iodine, a carrier liquid for holding the molecular iodine, and a water absorbing material. The water absorbing material limits the molecular iodine forming non-active species. The formulation allows the molecular

One embodiment of the present invention features a carrier liquid ethanol. The ethanol is preferably devoid of water, as in absolute alcohol. As used herein, the term “absolute alcohol” refers to alcohol with no or limited water content as such term is used in a commercial context. One formulation molecular iodine having a concentration in absolute alcohol in the range of 0.1 to 1.0 Gm molecular iodine in 13 milliliters of absolute alcohol. Molecular iodine saturates absolute alcohol at a concentration of 1 Gm per 13 milliliters of absolute alcohol. A further embodiment of the present formulation features a molecular sieve particle of between 2 and 4 A which captures and adsorbs water.

These and other features and advantages will be apparent from viewing the Drawings which are described in brief in the section below and reading the detailed description of the invention that follows.

### BRIEF DESCRIPTION OF THE DRAWING

FIG. 1 depicts a device embodying features of the present invention;

FIG. 2 depicts a schematic drawing having features of the present invention; and,

FIG. 3 depicts a schematic drawing having features of the present invention.

### DETAILED DESCRIPTION OF THE INVENTION

Embodiments of the present invention will now be described in detail with respect to a method and apparatus for administering an effective amount of molecular iodine (I₂) to treat oral disease in animals. The present description is considered to be the best mode to practice the invention with the understanding that the best mode may change over time. The description that follows is an exemplification of the present invention and should not be considered limiting. The device corresponds generally to the device of WO2012/118592, the content of which is incorporated by reference herein.

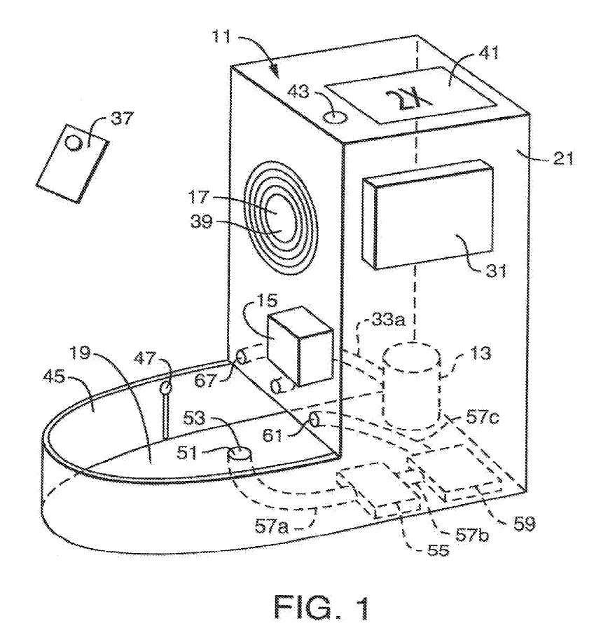

Turning now to FIG. 1, a device, embodying features of the present invention, is designated with the numeral 11. The device 11 is for administering a medicament, molecular iodine, for the prevention or treatment of a disease, bad oral breath in a dog [not shown in FIG. 1]. The device 11 comprises a source of medicament in a fluid 13, measuring means 15, an administration switch 17 and a water reservoir means 19, held in a housing 21.

The source of medicament 13 comprises a vessel containing molecular iodine in an ethanol solution. The molecular iodine is added to the water reservoir means 19 as an absolute alcohol solution saturated with I₂. An effective amount of this solution is formed with 0.001 to 1.0 ml, more preferably, 0.025 to about 0.05 ml to 0.1 ml. This effective amount in about 500 ml of water forms, if consumed by the animal is no more than about 250 to 500 micrograms of iodine daily, an amount that is safe and effective for thyroid function and an amount that substantially disinfects the oral cavity of an animal.

The dispensing means or measuring means 15 measures an amount of molecular iodine from the source of medicament or reservoir 13 to the water supply and in response to the dispensing signal, forming an aqueous solution having a concentration of molecular iodine not exceeding 1.0 micrograms per milliliter in the water reservoir means 19.

Sources of medicament 13 may take several forms, such as without limitation, tanks, conduits, pumps, bags, pouches, cartridges, vials, bottles, syringes and pressurized cans. Embodiments featuring a tank may incorporate the tank as an integral vessel that receives external fluid containing medicaments. Or, a vessel can be a replaceable consumable which is fitted to, or constructed and arranged to cooperate with conduits and/or pumps to communicate with measuring means. Pressurized cans and containers may not need pumps and use compressed air to propel the medicament. Although reference is made to pumps or pressurized vessels, the fluid may move from the source of medicament 13 to the measuring means 15 by gravity feed without pressure and without pumps. For the purpose of this description, the source of medicament is a glass vessel containing ethanol saturated with molecular iodine.

A further embodiment of the present invention is directed to a formulation for treating the oral cavity of an animal. The formulation comprises molecular iodine, a carrier liquid for holding the molecular iodine, and a water absorbing material. The water absorbing material limits the molecular iodine forming non-active species. The formulation allows the molecular iodine to be measured into water in a concentration not exceeding 1.0 micrograms per milliliter. This concentration represents the approximate limit that canines can detect. Concentrations above about 1.0 micrograms per milliliter are not readily accepted by canines.

The measuring means 15 is in fluid communication with the source of medicament 13 for measuring an aliquot of medicament corresponding to a dose for the prevention of plaque or treatment of a disease. As used herein, the term in “fluid communication” means plumbed together, or receiving fluid from one to the other. The measuring means 15 is also in communication with a water reservoir means and releases the aliquot into the water reservoir means 19 upon signal activation. The measuring means 15 comprises a timed or measured valve or pump, or metering syringe, or emptying a measuring vessel. For example, without limitation, where the medicament is held in a pressurized can, the measuring means may take the form of a dispensing valve [not shown] known in the art of the type used for dispensing aerosols and fluids. See for example: Remington: The Science and Practice of Pharmacy, 20^{th} Edition. Lippincott Williams and Wilkins (2000) at 976-978. As depicted and described herein, the measuring means is a timed pump plumbed to the source of medicament 13 via medicament conduit 33a.

The administration switch means 17 is in signal communication with the measuring means 15. Upon activation by a subject, such as a human or animal, administration switch means 17 produces an activation signal received by the measuring means 15.

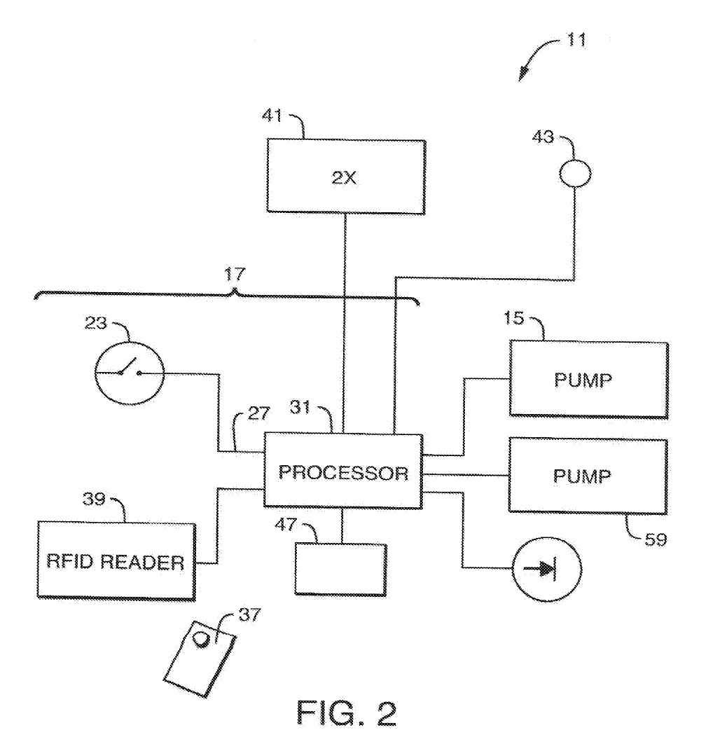

As best seen in FIG. 2 which depicts signal communication of device 11, administration switch means 17 has an on/off switch 23 for an electrical circuit 25 which operates measuring means 17 in the form of a pump [not shown] and a purge means 51 to be described more fully below. In the alternative, the administration switch means operates a motor or solenoid [not shown] with a mechanical link to a valve, measuring vessel, timed pump, metering syringe [not shown]. The switch 23 is further in signal communication with a CPU 31 which has timing functions and scheduling programming. The timing function times the power to the pump or solenoid such that the pump, valve, measuring vessel or metered syringe dispenses a desired dose of medicament in the water reservoir means 19. The scheduling function will dispense medicament at selected times of the day and for individual subjects from a population of potential subjects.

The administration switch means 17 of FIG. 1 and FIG. 2 further comprises a photometric or electromagnetic sensor which detects the presence of the subject. For example, without limitation, one embodiment of the present invention comprises a camera [not shown] and CPU, such as CPU 31, with visual recognition software for detecting the presence of an animal. As illustrated in FIG. 1 and 2, the photometric or electromagnetic sensor detects the presence of a tag 37. The tag 37 is recognized by sensor 39 by virtue of bar coding or other optical or electromagnetic coding. For example, one embodiment of the present invention features a sensor such as an optical scanner.

One embodiment, as illustrated, is an RFID tag 37. RFID tags are available from numerous vendors. A plurality of RFID tags 37, each coded separately for a plurality of individual subjects, are used to administer medicaments to selected or identified subjects from a population of potential subjects, at selected times or to receive selected medicaments. Tags and sensors which can detect a subject from a population of potential subjects are used in embodiments of the present invention denote an identified subject or identified animal. Different tags having different codes are used to denote one of more identified animals from the potential population or separate identified animals for different medicaments or doses.

Although a CPU 31 is depicted in FIG. 2, control means can comprise other analog and digital equipment which receives signals from the sensor and in response to the signal issues an activation signal to measuring means. Embodiments of the present invention feature administrative switching means having control means in the form of computer processing units (CPUs) integrated into a device 11. However, CPU 31 may be located apart from the device 11, as a separate computers, server, personal computers, internet linked computers and the like, in signal communication with measuring means by means of wired or wireless communication devices.

Embodiments of the present invention feature control means such as CPU 31 having memory to store information regarding selected subjects and the administration of medicaments. Such memory is known in the art and is not shown for purpose of clarity.

Embodiments further comprise timing elements or clock functions and programming to administer medicament in accordance with a schedule. Clock functions are common in CPU systems supporting CPU 31. Scheduling programming is also well known in the art. One embodiment of the present invention features molecular iodine administered to a selected subject or animal a schedule of once or twice daily. The doses are separated in time, for example eight to twelve hours. Programming is facilitated with a display screen 41 and controls 43 as best seen in FIG. 1.

The water reservoir means 19 is for receiving the aliquot and forming an aqueous solution of medicament. Water reservoir means 19 comprise a space constructed and arranged to receive a sized or shaped fluid receptacle, a fluid receptacle integral with the device or a disposable or removable cup, feeding and/or watering bowl or glass. As best seen in FIG. 1, the water reservoir means, as depicted, is integral with the device 19 and comprises a basin 45 for holding a suitable amount of water, for example 100 to 800 ml. Water reservoir means 19 has a volume sensor 47 that detects when the basin 45 is at capacity or empty. The volume sensor 47 is in signal communication with CPU 31 which coordinates the administration switch means 17 and measuring means 15 such that medicament is not added to the basin 45 when basin 45 cannot accommodated additional fluid or when the addition of medicament would not lead to an appropriate dilution.

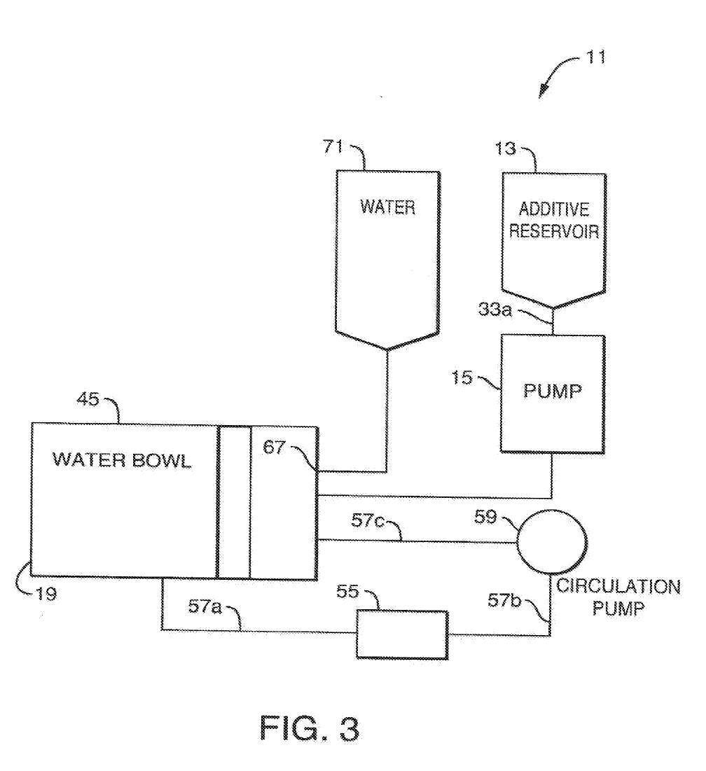

Water reservoir means 19 is in fluid communication with a source of water 71, best seen in FIG. 3 which depicts a fluid diagram of device 11. The flow of which is controlled by a valve or siphon mechanism [not shown] known in the art and under signal control of the volume senor 47 and CPU 31 or under hydrodynamic-mechanical control. Water flows into basin 45 via water inlet 67.

As depicted in FIG. 1, the device 11 further comprises purge means 51 in communication with the water reservoir means 19. Purge means 51 removes medicament from the basin 45. As depicted, purge means 51 comprises a drain 53, purge filter 55, purge conduits 57a-c, pump 59 and purge outlet 61. Liquid is removed from the basin 45 through drain 53 and conveyed by purge conduits 57a-c to a purge filters 55, purge pump 59 and on to the purge outlet 61. Thus, water is cleaned and medicament removed and reintroduced into the basin 45.

Purge filter 55 is made of porous materials to remove hair, dust and dirt. Purge filter 55 has an activated charcoal bed to remove medicament. Removal of medicament may be advantageous where the device 11 is used in the presence of small children.

Purge means 51 may also comprise waste receptacles [not shown] for storing a volume of water. Removal of the water may obviate the need for a purge filter 55. Purge means 51 can be constant or switched by a purge switch [not shown] which directs the liquids in the water reservoir means to adsorption materials or into the waste reservoir. For example, the purge pump 59 of purge means 51 is in signal communication with a purge switch means [not shown] and is actuated by a purge signal. The purge switch means comprises a timed electrical or timed mechanical switch that is coordinated with the administration switch means 17, or separate or combined with administration switch means 17, including incorporation of programming in control means such as a CPU 31. For example, without limitation, one control means is programmed issue a purge signal after an animal leaves the water reservoir.

The present device 11 is plumbed with a source of water shown in FIG. 3 which communicates with the basin 45 through a water inlet 67. Water inlet 67 releases water to fill basin 45 under the control of water level sensor 47 and CPU 31 or mechanical hydrodynamic means [not shown] known in the art.

Embodiments of the present invention directed to a method for administering a medicament to an animal will be described in detail with respect to FIG. 1 and device 11. The method comprising the steps of providing a device 11 having a source of medicament in a fluid 13, measuring means 15, an administration switch 17, and water reservoir means 19 as previously described. a subject, such as a house pet, is fitted with a tag 37. As the subject approaches the water reservoir means 19, the tag 37 is recognized by administration switch means 17 and measuring means 15 is activated with an administration signal to dispense a dose of medicament such as molecular iodine. Administration switch means 17 recognizes the time the subject leaves the water reservoir means 19, and CPU 31 issues a purge signal to purge means 51 to remove medicament form the water reservoir means 19.

Embodiments of the present method are well suited for the administration of molecular iodine to the oral cavity of a subject such as a human or an animal. One medicament is a saturated iodine in ethanol comprising iodine crystals and absolute ethanol. A saturated solution of molecular iodine in absolute alcohol comprises 1 Gr of molecular iodine in 13 ml of absolute alcohol. In the alternative a 10% solution is prepared by dissolving 1 gram of iodine crystals in 100 ml of absolute ethanol. A 1% solution is prepared from a1:10 dilution of the 10% solution of iodine in absolute ethanol. A further embodiment of the present formulation features a molecular sieve particles of between 2 and 4 A which captures and adsorbs water.

The method of treating an animal for an oral disease will now be described with respect to the operation of the apparatus. As an animal approaches the apparatus, it presence is detected and the apparatus dispenses molecular iodine in an aqueous solution in a concentration of 1.0 to 0.01 micrograms per milliliter in the bowl 45 The animal is allowed the animal to drink of the solution. The solution kills bacteria forming plaque or bacteria contributing to halitosis or other oral pathogens.

For acute treatments a concentration of 1.0 to 0.5 micrograms per milliliter may be used. These and other features are more fully developed in the Examples.

### EXAMPLES

### Example 1

This example demonstrates a concentration of molecular iodine [I₂] added to water to a final concentration of 0.5 micrograms/mL to 1.0 microgram/mL will significantly reduce plaque formation and significantly reduce or eliminate halitosis in animals. All studies described here were conducted with adult dogs.

First, a double-blind, placebo controlled clinical trial was conducted using 20 adult dogs of mixed breed and weights. The dogs were randomly divided into two groups. Group 1 dogs of mixed breed and weights. The dogs were randomly divided into two groups. Group 1 dogs were further divided into two groups of five dogs. Five received molecular iodine [I₂] dissolved into 1000 mL water to a final concentration of 0.5 microgram/mL. The second group of five dogs received molecular iodine [I₂] dissolved into 1000 mL water to a final concentration of 1.0 microgram/mL. The placebo group of 10 dogs received no iodine. The stock solution of molecular iodine [I₂] was prepared by weighing 0.1 gram solid iodine crystals and adding to 100 mL 100% ethyl alcohol with essentially all contaminating water e.g., 0.4% sequestered or bound into and on 3A silica particles. The resulting stock solution had a final concentration of 0.1% molecular iodine [I₂]. When 1.0 mL of the stock 0.1% molecular iodine [I₂] is added to 1000 mL water the final concentration of molecular iodine [I₂] in water is 1.0 microgram/mL. Consequently, when 0.5 mL of the stock 0.1% molecular iodine [I₂] is added to 1000 mL water the final concentration of molecular iodine [I₂] in water is 0.5 microgram/mL.

The clinical trial samples containing either drug i.e., molecular iodine [I₂] or placebo i.e., 100% ethyl alcohol were prepared as described below. Twenty mL borosilicate scintillation vials with polypropylene closures in the cap were used to prepare each sample for delivery into the 1000 mL water sample. The prescribed protocol is 28 days. Each dog receives one dose of drug or placebo each day first thing in the morning. Therefore, 20 boxes containing 28 vials of either drug or placebo are required. Ten of the boxes contained drug, 5 containing 1.0 mL of 0.1% molecular iodine [I₂] and 5 containing 0.5 mL of 0.1% molecular iodine [I₂]. All 28 vials in each study box vials were then filled to a total volume of 10 mL using 100% ethyl alcohol. Therefore, 10 study boxes contained a total of 280 vials with drug. Similarly, the placebo study boxes were prepared in the same way. The difference is that all 10 study boxes containing 280 vials in the placebo group contained only 10 mL 100% ethyl alcohol with no iodine.

Each dog was weighed, blood drawn for determination of thyroid level and a qualitative assessment was made for halitosis using a 0, 1, 2, 3, 4 and 5 scale with 0 indicating no discernible “doggie breath produced by hydrogen sulfide H₂S” and 5 being very noxious, strong odor. Dogs were anesthetized, marginal tooth-gum line measurements were taken for proscribed teeth (required for veterinary oral health counsel certification of efficacy at reducing plaque in dogs found at www.VOHC.com) and dog teeth were cleaned to remove all plaque and tarter. All dogs were given either drug or placebo each morning for 28 days. Journal entries were kept for each dog to monitor for compliance, observations regarding dog drinking behavior and comments regarding dog breath/odor. Once each dog drank all the water i.e., 1000 mL the water bowl was filled with fresh water. The following day all the remaining water was discarded and the clinical trial sample was added to the water bowl. At the end of the study i.e., 28 days each dog was sedated, breath was evaluated and teeth were stained with disclosing solution that is proven to stain plaque red. The attending veterinarian who measured the initial marginal tooth-gum line measured all the proscribed teeth using the published technique (Scherl D S, Coffman L, et al. Validation of a New Dental Plaque Quantification Method in Dogs. J Vet Dent 24(1); 14-20, 2007). All values from all 20 dogs were recorded and whole mouth scores were calculated.

The statistical evaluation and the report is summarized below. Despite some variability in scoring and minor compliance issues, there did appear to be a statistically significant reduction in whole mouth plaque scores from Day 0 to Day 28 in Treated dogs compared to Placebo dogs. Additionally, dog size did not appear to play a role in predicting plaque score outcome. As noted, T4 levels remained within normal range after treatment. Furthermore, there was no difference between mean plaque score for dogs receiving 0.5 microgram/mL versus dogs receiving 1.0 micrograms/mL. In conclusion, the Iodine treatment appeared safe and effective for the prevention of plaque in this study.

The halitosis secondary end point was more complicated since the attempt was made to use an Interscan Corporation Halimeter® that quantitatively measures volatile sulfur compounds (VSC) in the oral cavity. The Halimeter was designed for use by dentists and humans. One of the requirements to obtain reliable readings is for the conscious human subject to blow into a straw connected into the Halimeter. The problem encountered with dogs while under anesthesia is obtaining a valid reading since they are unable to “blow” into the straw. Early dog readings were erratic and turned out to be unreliable. However, all dogs with 4-5 odor e.g., on a 0-5 scale had very high relative VSC readings. These dog oral breath odors were confirmed qualitatively by observers e.g., dental technicians. At the conclusion of the clinical trial attempts were made to obtain readings on 5 dogs with high Halimeter VSC levels e.g., 4-5 and 2 of the 5 had similar VSC levels and 3 of the 5 had very reduced VSC levels e.g., 0-1. Interestingly, the three dogs with lower VSC levels compared to their initial VSC levels were in the iodine group and the 2 with high VSC levels were in the placebo group. While not statistically significant these data are persuasive especially when compared to the qualitative odor assessments both by the attending dental technicians and the dog owners as recorded in their clinical trial record log books.

First, the five dogs cited above had qualitative halitosis measurements that were the same as the quantitative Halimeter readings e.g., the three dogs with lower VSC levels compared to their initial VSC levels were in the iodine group and the 2 with high VSC levels were in the placebo group. Second, 11 study dogs with qualitative odor measurements i.e., smell that were either recorded in the clinical trial log books or cited by the attending dental technicians indicated that 5 iodine-treated dogs had reduced odor e.g., initial smell was 3-5 and final odor was 0-1. The other 6 dogs had no change in their odor scores e.g., 3-5 initially and 3-5 at the conclusion of the study. The remaining 3 dogs had no data that could be interpreted. In summary, 8 iodine-treated dogs had reduced oral smell or doggie breath or VSC/H₂S. Two of the iodine-treated dogs had either no reading or measurement or no change in odor. In contrast, 8 of the ten dogs in the placebo group had no reduction in oral bad breath/VSC/doggie breath.

Molecular iodine [I₂] is an effective antimicrobial oral rinse for animals and dogs especially and that dog halitosis could be reduced or eliminated by using molecular iodine [I₂] to kill the bacterial responsible for hydrogen sulfide i.e., H₂ S production in the sulcus between the gum and tooth in an anaerobic ecosystem with other microbes. However, molecular iodine also participates in a chemical reaction with hydrogen sulfide [H₂S], a gas. The reaction shown below occurs rapidly at temperatures between 20-60° C. including the oral cavity of animals and dogs in particular. The reaction is shown below (1).

H₂S+I ₂−>HSI+HI (1)

The most significant result of this chemistry is that molecular iodine [I₂] neutralizes volatile hydrogen sulfide gas [H₂S] to a non-volatile, aqueous form. Therefore, molecular iodine [I₂] has a dual effect on the problem of halitosis in animals and dogs in particular. Namely, molecular iodine [I₂] kills the sulfur-producing bacteria responsible for hydrogen sulfide production and molecular iodine [I₂] neutralizes volatile hydrogen sulfide gas-bad breath/odor. The microorganism responsible for halitosis is Solobacterium moorei. Perhaps more significant is that many new studies implicate the surface of the tongue as harbingers of malodor-producing bacteria. Microbial and topographic distribution of bacterial types and loads associated with mid-morning oral malodor on the tongue surface have been conducted and a wide variety of microbes have be implicated. Our data suggests that molecular iodine [I₂] kills all pathogens implicated in tongue-specific halitosis. Therefore, molecular iodine [I₂] may represent the most effective oral treatment for halitosis in animals and dogs specifically.

### Example 2

Molecular iodine [I₂] has an odor and taste in water depending on the concentration. Early studies indicated that 10 micrograms/mL was 100% unacceptable to dogs and cats. They would not drink iodine in their water at this concentration. Dogs can detect odors 10,000-100,000 more effectively than humans. Stated differently, dogs can detect some odors in parts per trillion. Therefore, experiments were designed to test dog drinking behavior when presented with various concentrations of molecular iodine [I₂] dissolved in water. Identical stainless steel bowls containing one gallon water were prepared. Seven identical bowls of water were placed on the floor in the usual place for dog water.

The bowls contained the following concentrations of molecular iodine [I₂]:

1—no iodine
2—0.1 microgram/mL
3—0.5 microgram/mL
4—1.0 microgram/mL
5—2.0 microgram/mL
6—4.0 microgram/mL
7—8.0 microgram/mL

Three dogs were able to roam and drink at will. The test water bowls were the only water source available. The results indicated that no water was consumed for bowls 5, 6 and 7. All the water in bowls 1, 2, 3 and 4 was consumed. This experiment was repeated 3 times with exactly the same result. Dog preference is clearly proven and 1.0 microgram/mL is the greatest concentration of molecular iodine [I₂] that dogs will freely drink without restriction.

Thus, we have described embodiments of the present invention in detail with the understanding that the description herein can be modified and altered by those skilled in the art without departing from the teaching herein. Thus, the present invention should not be limited to the precise details herein but should encompass the subject matter of the claims that follow and their equivalents.

### Results of Clinical Trials

Two double-blind, placebo-controlled clinical trials were conducted to demonstrate efficacy of adding elemental iodine into dog drinking water to reduce plaque formation on teeth. Plaque formation leads to gingivitis and periodontal disease and often progresses to systemic disease and death in dogs. Clinical trial 1 was conducted at a humane shelter with all dogs enclosed in kennels using a manually added drug vs. placebo. Subsequently, Clinical Trial 2 was conducted at a humane shelter with all dogs enclosed in kennels but in this case the iodine vs placebo was administered via an automated system.

Preliminary experiments with dogs proved that dogs would not drink elemental iodine in dog drinking water if the concentration of elemental iodine was too high e.g., 5 micrograms/mL. The inventor evaluated a variety elemental iodine concentrations e.g., 0.5, 1.0, 2.0, 3, 5, 10, 15 and 20 micrograms/mL and discovered that dogs preferred drinking water with elemental iodine below 2 micrograms/mL. We evaluated the ability of elemental iodine to kill oral bacteria at a concentration of 2 micrograms/mL responsible for plaque formation and discovered that elemental iodine was effective at this dose i.e., microbial kill >3 logs/min. We evaluated lower concentrations of elemental iodine e.g., 0.05, 0.1 and 0.5 micrograms/mL. Our test dogs never rejected these iodine concentrations. We hypothesized that molecular iodine concentrations below 0.5 micrograms/mL may not be not effective in killing oral bacteria responsible for plaque formation. We confirmed this assumption by performing microbial analysis. The inventor sent samples of two part preparations e.g., 1 vial with water and a second vial with iodine in 100% ethyl alcohol. These were combined before analysis so that the final concentrations were 0.05, 0.1, 0.5, 1.0, 2.0 and 5.0 micrograms/mL. The test organism was a known plaque-producing bacterium i.e., Neisseria spp. A contract research laboratory in Texas i.e., MicroChem Laboratories performed the use dilution experiments. Iodine concentrations at 0.05 and 0.1 micrograms/mL demonstrated no antimicrobial efficacy. The 0.5 microgram/mL iodine demonstrated a 100-fold reduction in microbial concentration at the 1 minute time point. The 1.0 microgram/mL iodine demonstrated a 1000-fold reduction at the 1 minute time point and 2.0 and 5.0 microgram/mL demonstrated >4 logs kill at the 1 minute time point.

We conducted two double-blind, placebo-controlled clinical trials using both 0.5 and 1.0 micrograms elemental iodine in water. We proved that 0.5 and 1.0 micrograms/mL elemental iodine in dog drinking water were statistically significantly identical in preventing plaque formation in all dogs regardless of size or age. In addition, we proved that elemental iodine could be added to dog drinking water either manually or via an automated device and the observed plaque prevention could be achieved.

Dogs often rejected drinking elemental iodine concentrations above 2 micrograms/mL and elemental iodine concentrations below 0.5 micrograms/mL were not effective in killing oral bacteria based on independent use dilution experiments.

In summary, the iodine administration to dog drinking water for plaque prevention was proven effective at both 0.5 and 1.0 micrograms/mL water. Furthermore, neither dog weight or age affected the plaque prevention effect. Finally, the iodine added to dog drinking water had no effect on dog thyroid hormone levels.

### Excluded Dosage Ranges

To be effective, the dose range must be between 0.5-2.0 micrograms of molecular iodine per mL of water. Doses below 0.5 micrograms/mL are ineffective in preventing plaque and halitosis; doses above 2.0 micrograms/mL are rejected by most pets (the pet refuses to drink). Doses are administered by adding quantities of either a 1% or 10% (or other stock solution) of molecular iodine in absolute alcohol to a pet's drinking water making sure that the daily consumption of iodine never exceeds 500 micrograms per day.

Thus, the molecular iodine dose range cannot be below 0.5 micrograms/mL of water, and the dose range cannot be above 2.0 micrograms/mL of water.

Therefore, it is an object of the present invention to supply a dose of molecular iodine to a pet on a daily basis where an individual dose is not below 0.5 micrograms of molecular iodine per mL of water and not above 2.0 micrograms of molecular iodine per mL of water with the constraint that the pet must not receive more than 500 micrograms of molecular iodine per day.

Several descriptions and illustrations have been presented to aid in understanding the present invention. One with skill in the art will realize that numerous changes and variations may be made without departing from the spirit of the invention. Each of these changes and variations is within the scope of the present invention.

1. A method of treating plaque and halitosis in an animal comprising:
preparing a stock iodine solution made by dissolving a ratio of iodine crystals in absolute alcohol, and then adding an amount of said stock iodine solution or a further dilution of said stock solution to a particular quantity of the animal's drinking water resulting in a dose concentration of greater than or equal to 0.5 microgram per mL, but not less than 0.5 microgram per mL, and less than or equal to 2.0 micrograms per mL, but not greater than 2.0 microgram/mL of molecular iodine in the drinking water
automatically recognizing a particular animal from a population of animals and adjusting the dose concentration according to a stored dose concentration for that particular animal, limiting a total dose for a particular animal to 500 micrograms of iodine per day;
allowing the particular animal to drink from said drinking water.

2. The method of claim I wherein said dose is made as the animal approaches a solution dispensing station.

3. The method of claim I wherein said animal is provided a supply of water over time, and said solution is purged and generated over such time.

4. The method of claim 1 wherein the particular animal wears and RFID tag, and the step of automatically recognizing is a recognition of said RFID tag.

5. The method of claim 1 wherein the particular animal is automatically recognized by comparing a camera image with a stored image of the particular animal.

6. A method for controlling plaque and halitosis in pets comprising
dispensing a dose of molecular iodine into a pet's drinking water forming a aqueous solution having a concentration of molecular iodine between 0.5 micrograms/mL of water, but not less than 0.5 micrograms/mL and 2.0 micrograms/mL of water, but not more than 2.0 micrograms/mL of water.

7. The method of claim 6 wherein a sensor senses when the pet approaches the drinking water.

8. The method of claim 7 wherein the dose is dispensed into the drinking water when the pet approaches the drinking water.

9. The method of claim 8 wherein the pet is identified as a particular pet from a community of pets.

10. The method of claim 6 wherein the pet is not given more than 500 micrograms of molecular iodine per day.

11. The method of claim 6 wherein the pet is a dog.

12. The method of claim 6 wherein the drinking water is purged of iodine when the pet departs from proximity of the drinking water.

13. A method of treating plaque and halitosis in a dog comprising:
preparing a stock iodine solution of molecular iodine of known concentration by dissolving a particular amount of molecular iodine in absolute alcohol;
dispensing an aliquot of said stock iodine solution, or a dilution of the stock iodine solution, into a predetermined quantity of the dog's drinking water, said aliquot chosen to result in an iodine concentration in water of between 0.5 micrograms/mL, but not less than 0.5 micrograms/mL and 2.0 micrograms/mL, but not more than 2.0 micrograms/mL;
automatically recognizing a particular dog from a population of dogs and adjusting the final concentration according to a stored concentration for that particular dog, limiting a total dose for the particular dog to 500 micrograms of iodine per day;
allowing the particular dog to drink from the drinking water after said dispensing.

14. The method of claim 13 wherein said aliquot is dispensed as the dog approaches a pet drinking station.

15. The method of claim 14 wherein the drinking water is purged when the particular dog departs from proximity to said pet drinking station.

16. The method of claim 13 wherein the particular dog wears and RFID tag, and the step of automatically recognizing is a recognition of said RFID tag.

17. The method of claim 13 wherein the particular dog is automatically recognized by comparing a camera image with a stored image of the particular dog.
